(19) United States
(12) Patent Application Publication
(10) Pub. No.: US 2025/0369949 A1
(43) Pub. Date: Dec. 4, 2025

(54) WATER/OIL EMULSION SENSOR AND DOWNHOLE TOOL INCLUDING SUCH SENSORS

(71) Applicant: OPENFIELD, Versailles (FR)

(72) Inventors: Eric DONZIER, Bercheres sur Vesgre (FR); Linda ABBASSI, Katy, TX (US); Emmanuel TAVERNIER, Paris (FR)

(21) Appl. No.: 19/224,718

(22) Filed: May 31, 2025

(30) Foreign Application Priority Data
May 31, 2024 (FR) FR2405685

(51) Int. Cl. G01N 33/28; G01N 27/22; G01N 27/74

(52) U.S. Cl. CPC G01N 33/2847; G01N 27/22; G01N 27/74

(57) ABSTRACT

A water/oil emulsion sensor is sensitive to a water content and an oil content of a multiphase fluid mixture flowing as an emulsion in a hydrocarbon well. The sensor includes a conductive tip at a front part, a hollow isolating body at a middle part and a hollow conductive body at a back part, said front part having a conical shape, said middle and back parts having a substantially cylindrical shape, said parts of said sensor being coaxial, extending along a longitudinal axis and being assembled in a sealed manner, an inductive module housed in the hollow isolating body and comprising an emission coil and a detection coil, a capacitive module housed in the hollow isolating body and comprising a conductive cylindrical surface, and an electronic unit comprising a processing module coupled to an inductive power supply and measurement module connected to the inductive module, and to a capacitive power supply and measurement module connected to the capacitive module.

### TECHNICAL FIELD

The invention relates to a water/oil emulsion sensor sensitive to a water content and an oil content of a multiphase fluid mixture flowing in a hydrocarbon well. In an emulsion, one liquid (the dispersed phase) is dispersed in the other (the continuous phase). In a multiphase fluid mixture flowing in a hydrocarbon well, there may be different types of emulsions. In particular, oil and water may form an oil-in-water emulsion (i.e. oil is the dispersed phase, and water is the continuous phase), or a water-in-oil emulsion (i.e. water is the dispersed phase and oil is the continuous phase). A downhole tool, for example a production logging tool, including at least one water/oil emulsion sensor is used to analyze the multiphase fluid mixture flowing from a hydrocarbon bearing zone into the hydrocarbon well. Such a downhole tool is particularly adapted to be deployed in hydrocarbon wells comprising vertical well sections, deviated well sections, substantially horizontal well sections or a combination of the above.

Such downhole tools typically operate in the harsh downhole environment of hydrocarbon wells at downhole pressure (typically in the range of one hundred to 2000 bars) and temperature (typically in the range of 50 to 200° C.) conditions, and in possibly corrosive fluids.

### BACKGROUND

During the production of hydrocarbon wells, it is necessary to monitor various characteristic parameters, like the relative volumetric flow rates of the different phases (e.g. oil, gas and water) of the multiphase fluid mixture flowing into the pipe of the well from the hydrocarbon bearing zones. Further, current hydrocarbon wells often comprise a vertical well section, deviated well sections, highly deviated well sections and even substantially horizontal well sections. The interpretation of the flow in such complex wells is challenging because small changes in the well deviation and the flow regime influence the flow profile. Thus, an accurate monitoring requires sensors or probes capable of imaging a surface section or a volume section of the pipe and providing an estimation of the surface section or the volume section occupied by each phase.

Production logging of hydrocarbon wells (e.g., oil and gas wells) faces numerous challenges related to the complexity of the multiphasic flow conditions and the harshness of the downhole environment.

Gas G, oil O, water W, mixtures O&W flowing in wells, being either openhole or cased hole wells, may flow as an emulsion depending on the relative proportions of phases (“holdup”), their velocities, densities, viscosities, as well as pipe dimensions and well deviations. In order to achieve a good understanding of the individual phases flowrates and determine the relative contributions of each zone along the well, an accurate mapping of fluids types and velocities is required over the whole section of the hole (openhole well portion) or pipe (cased well portion) at different depths (i.e., the measured depth, defined on the basis of the distance traveled by the logging equipment along the well from its location to the surface, is different from the true vertical depth and generally longer than true vertical depth, due to deviations in the well from the vertical). Further, production issues greatly vary depending on reservoir types and well characteristics resulting in the need for a flexible production logging technology working with different types of sensing physics. For example, in multiphase fluid mixtures flowing in hydrocarbon wells, often oil and water are flowing as a water/oil emulsion, namely a liquid dispersed phase dispersed in a liquid continuous phase. There may be different types of water/oil emulsions. As an example, oil may be the dispersed phase, and water may be the continuous phase, oil and water forming an oil-in-water emulsion. As another example, water may be the dispersed phase and oil may be the continuous phase or a water-in-oil emulsion. An emulsion is a pseudo-homogenous fluid. Typically, the dispersed phase may be approximated by spherical structure well below millimeter, for example of the micron order and therefore cannot be measured by usual sensors like local electric sensors or even optical sensors. There is a need for a sensor allowing measurement of emulsions whatever the nature of the continuous phase (being oil or water) and the dispersed phase (being water or oil).

Furthermore, high pressure, up to 2000 bars, high temperature, up to 200° C., corrosive fluid (H2S, CO2) put constraints on sensors and tool mechanics.

Furthermore, solids presence in flowing streams can damage equipment. In particular, the sand entrained from reservoir rocks will erode parts facing the fluid flow. Solids precipitated from produced fluids due to pressure and temperature changes, such as asphaltenes, paraffins or scales create deposits contaminating sensors and/or blocking moving parts (e.g., spinners).

Furthermore, the tool deployment into the well can be difficult and risky. In highly deviated or horizontal wells, tools must be pushed along the pipe using coiled tubing or pulled using tractor which is difficult when tools are long and heavy. Pipes may be damaged by corrosion or rock stress which may create restrictions and other obstacles. During the logging operation, equipment can be submitted to high shocks. Thus, in such environments, it is highly preferable to have light and compact tools.

Furthermore, the cost is also an important parameter in order to provide an economically viable solution to well performance evaluation even in mature fields having low producing wells in process of depletion with critical water production problems.

### SUMMARY OF THE DISCLOSURE

It is an object of the invention to propose a water/oil emulsion sensor allowing measurement of multiphase fluid mixtures flowing as emulsions in hydrocarbon wells whatever the nature of the continuous phase (being oil or water) and the dispersed phase (being water or oil). It is a further objective to design a water/oil emulsion sensor that is compact, accurate and reliable. It is a further objective to integrate at least one of such a water/oil emulsion sensor in a downhole tool, for example a production logging tool that is structurally simple and reliable to operate whatever the downhole conditions.

According to one aspect, there is provided a water/oil emulsion sensor sensitive to a water content and an oil content of a multiphase fluid mixture flowing as an emulsion in a hydrocarbon well, the water/oil emulsion sensor including:
a conductive tip at a front part, a hollow isolating body at a middle part and a hollow conductive body at a back part, said front part having a conical shape, said middle and back parts having a substantially cylindrical shape, said parts of said sensor being coaxial, extending along a longitudinal axis and being assembled in a sealed manner,
an inductive module housed in the hollow isolating body and comprising an emission coil and a detection coil,
a capacitive module housed in the hollow isolating body and comprising a conductive cylindrical surface, and
an electronic unit comprising a processing module coupled to an inductive power supply and measurement module connected to the inductive module, and to a capacitive power supply and measurement module connected to the capacitive module,
the inductive power supply and measurement module is arranged to inject an excitation inductive current into the emission coil such that if the multiphase fluid mixture is conductive, a first induced inductive current is generated into a first investigation zone surrounding the inductive module and a second induced inductive current proportional to the first induced inductive current is measured in the detection coil, the second induced inductive current being representative of the water content within the multiphase fluid mixture in the first investigation zone,
the capacitive power supply and measurement module is arranged to apply an excitation capacitive voltage to the conductive cylindrical surface and to measure a capacitance of the multiphase fluid mixture in a second investigation zone surrounding the conductive cylindrical surface, the capacitance being representative of the oil content within the multiphase fluid mixture in the second investigation zone,
the inductive module is arranged in a nested and proximate manner relatively to the capacitive module within the hollow isolating body at the middle part, the emission coil and the detection coil being in-line and extending according to the longitudinal axis, the conductive cylindrical surface being coaxial to the longitudinal axis, concentric and surrounding both emission and detection coils such that the first and second investigation zones substantially overlap each other so that water content and an oil content of the multiphase fluid mixture flowing as emulsion are determinable at a substantially identical location.

The conductive tip and the hollow conductive body may be made of a metallic alloy withstanding well conditions, and the hollow isolating body may be made of ceramic.

Each of said emission coil and detection coil may comprise a core made of a ferrite hollow cylinder or a toroid (square toroid or circular toroid), and a winding including multiple turns of a metallic wire. The winding may include from a few turns (for example eight) to a few dozen of turns (for example thirty) of said metallic wire.

The inductive power supply and measurement module may be arranged to inject an excitation inductive current having an amplitude ranging from 0.1 mA to 20 mA and a frequency ranging from a few kHz (for example 1 kHz) to a few hundreds of kHz (for example 900 kHz) into the emission coil. As examples, the excitation inductive current may be a sinusoidal, square or triangle periodic signal.

The capacitive power supply and measurement module may be arranged to apply a sinusoidal excitation capacitive voltage having an amplitude ranging from 100 mV to 9V and a frequency ranging from 1 kHz to 900 kHz to the conductive cylindrical surface.

The conductive cylindrical surface may be supported onto a supporting tube, said conductive cylindrical surface being made of metal, said supporting tube being made of plastic or ceramic material.

The conductive tip may be connected to a ground potential by means of a tip wire, the tip wire closing a loop between the conductive tip and the hollow conductive body.

The water/oil emulsion sensor may further comprise a connector positioned at the back of said sensor so as to close the hollow conductive body at a side of the sensor opposite to the conductive tip.

The processing module may be arranged to extract complex impedance values in angular phase and quadrature from complex inductor impedance values provided by the inductive power supply and measurement module connected to the inductive module combined with complex capacitor impedance values provided by the capacitive power supply and measurement module connected to the capacitive module and to estimate water and oil content based on an experimental model fitted for example by a polynomial approximation (e.g. a polynomial of order n).

The water/oil emulsion sensor may further comprise an active counter electrode having a first part and a second part on each side of the conductive cylindrical surface, each of said part being a ring having essentially the same diameter than the conductive cylindrical surface, both parts being connected to a reverse voltage power supply suppling the active counter electrode with a reverse voltage compared to an excitation capacitive voltage applied to the conductive cylindrical surface by the capacitive power supply and measurement module.

According to a further aspect, there is provided a downhole tool comprising an elongated cylindrical body of longitudinal axis, said body carrying a centralizer arrangement comprising articulated centralizer arms, said arms being operable from a retracted configuration into a radially extended configuration, wherein at least one arm carries at least one water/oil emulsion sensor of the invention.

Alternatively, the downhole tool may comprise an elongated cylindrical body of longitudinal axis, the body comprising at least one slot partially opening externally to accommodate at least one water/oil emulsion sensor of the invention.

Multiple water/oil emulsion sensor may be connected to a main processing module such as to form an array of sensors.

The downhole tool may further include other downhole fluid properties analysis probe of any type, chosen among the group comprising sensors responsive to physical parameters such as pressure, temperature, density, viscosity, refractive index, fluid velocity, gas bubble counts and holdups, fluorescence, spectroscopic absorption of the multiphase fluid mixture.

The water/oil emulsion sensor results in a simple and compact structure achieving a high accuracy, yet at a low cost, together with simple operation and maintenance. Each local sensor enables measuring the water content and the oil content simultaneously and in a well-defined measuring area of reduced size (i.e. essentially in the same place) of the multiphase fluid mixture flowing as an emulsion in the hydrocarbon well, and, thus, the water content and the oil content of the multiphase fluid mixture flowing in the hydrocarbon well with a very good resolution. The downhole tool of the invention enables deploying a single local water/oil emulsion sensor or an array of local water/oil emulsion sensor whatever the orientation of the well section.

Other advantages will become apparent from the hereinafter description of the invention.

### BRIEF DESCRIPTION OF THE DRAWINGS

The present invention is illustrated by way of examples and not limited to the accompanying drawings, in which like references indicate similar elements:

FIG. 1 is a side partial cross-sectional view illustrating a downhole tool comprising a water/oil emulsion sensor of the invention within a horizontal well section in a fully deployed configuration;

FIG. 2 is a side partial cross-sectional view illustrating a downhole tool comprising a water/oil emulsion sensor of the invention within a vertical well section;

FIG. 3 is a side partial cross-sectional view illustrating a water/oil emulsion sensor of the invention according to a first embodiment and a corresponding operating principle;

FIG. 4 is a side partial cross-sectional view illustrating a water/oil emulsion sensor of the invention according to a second embodiment and a corresponding operating principle;

FIG. 5, FIG. 6, FIG. 7 and FIG. 8 are, respectively, a side partial cross-sectional view, a one side cross-sectional perspective view, an exploded perspective view and an assembled perspective view illustrating the practical implementation of the water/oil emulsion sensor of the invention;

FIG. 9 and FIG. 10 are diagrams illustrating the response of the water/oil emulsion sensor of the invention in water/oil emulsions used to infer the water content and oil content of a multiphase fluid mixture flowing in a hydrocarbon well;

FIG. 11, FIG. 12 and FIG. 13 are, respectively, a one side perspective view, a side cross-sectional view and a front view illustrating various embodiments of a downhole tool including an array of water/oil emulsion sensors of the invention; and

FIG. 14 is a one side perspective view illustrating another embodiment of a downhole tool including multiple water/oil emulsion sensors of the invention.

### DETAILED DESCRIPTION

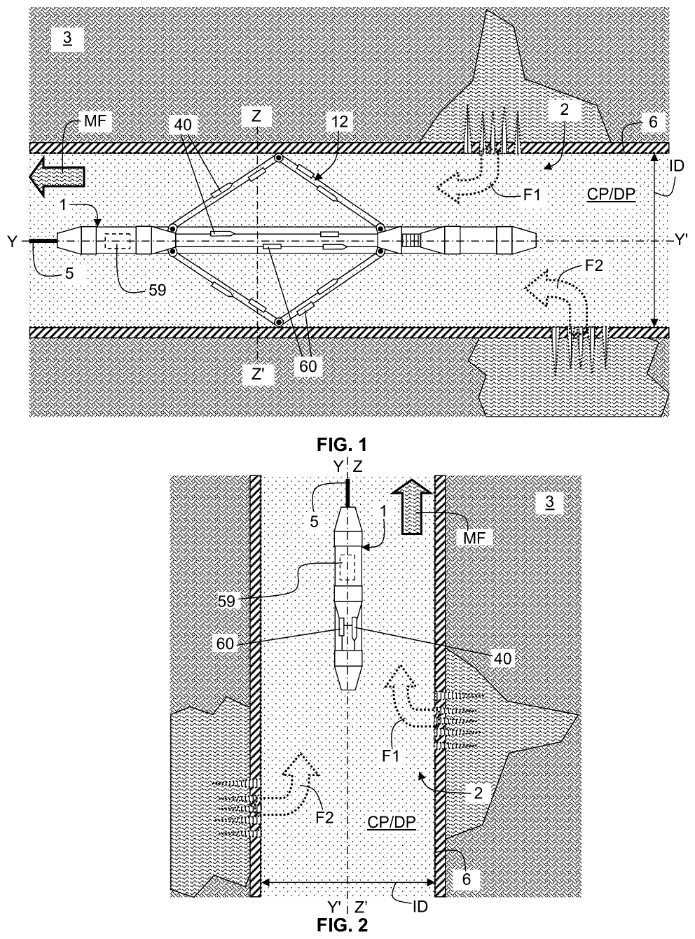

FIG. 1 and FIG. 2 illustrate a downhole tool 1, for example a production logging tool, being deployed into a wellbore of a hydrocarbon well 2 that has been drilled into a subterranean formation 3. In the particular example of FIG. 1, the downhole tool is deployed in a horizontal section of a hydrocarbon well. In the particular example of FIG. 2, the downhole tool is deployed in a vertical section of a hydrocarbon well. In both examples, said sections have been further fractured at defined locations (i.e. fracture clusters). The downhole tool 1 is used to analyze at least one property of a multiphase flow mixture MF flowing in the hydrocarbon well 2. The multiphase flow mixture MF is characterized by holdup, slippage velocity and phase segregation. Holdup is the percentage by volume of the gas, oil and/or water content in the wellbore measured over a cross-sectional area (based on the wellbore inner diameter ID). Slippage velocity is the relative velocity existing between light phases and heavy phase. Phase segregation is the tendency of fluids to stratify into different layers because of differences in density between oil O, water W and gas G and due to the immiscibility of water and oil, and the limited miscibility (depending on temperature and pressure) of gas in oil and water. Further, oil and water may also flow as a water/oil emulsion, wherein a liquid dispersed phase DP (oil or water) is dispersed in a liquid continuous phase CP (water or oil). There may be different types of water/oil emulsions. As a first example, oil and water may form an oil-in-water emulsion (oil being the dispersed phase and water being the continuous phase). As a second example, oil and water may form a water-in-oil emulsion (water being the dispersed phase and oil being the continuous phase). The wellbore refers to the drilled hole or borehole, including the open hole or uncased portion of the well. The borehole refers to the inside diameter of the wellbore wall, the rock face that bounds the drilled hole. The open hole refers to the uncased portion of a well. While most completions are cased, some are open, especially in horizontal or highly inclined wells where it may not be possible to cement casings efficiently. The downhole tool 1 is suitable to be deployed and run in the wellbore of the hydrocarbon well 2 for performing various analysis of the multiphase flow mixture MF properties irrespective of a cased or uncased nature of the hydrocarbon well. The downhole tool 1 may comprise various subsections having different functionalities and may be coupled to surface equipment through a wireline 5 (or alternative equipment such as coiled tubing adapted to displace the tool in horizontal and highly deviated wells) which is operable at a surface equipment to displace the tool along the well. At least one subsection comprises a measuring device generating measurements logs, namely measurements versus depth measured along the well or time, or both, of one or more physical quantities in or around the well 2. Wireline logs are taken downhole, transmitted through the wireline 5 to surface and recorded there, or else recorded downhole and retrieved later when a logging instrument is brought to surface. There are numerous log measurements (e.g. electrical properties including conductivity at various frequencies, sonic properties, active and passive nuclear measurements, dimensional measurements of the wellbore, formation fluid sampling, formation pressure measurement, flow rate measurements, etc . . . ) possible while the production logging tool 1 is displaced along and within the hydrocarbon well 2 drilled into the subterranean formation 3. Ancillary surface equipment is neither shown nor described in detail herein. In the following the wall of the wellbore irrespective of its cased (cement or pipe) or uncased nature is referred to wall 6. Various fluid (that may include solid particles) entries F1, F2 may occur from the subterranean formation 3 towards the wellbore 2. Once in the wellbore 2, these fluid entries form the multiphase flow mixture MF that generally flows towards the surface.

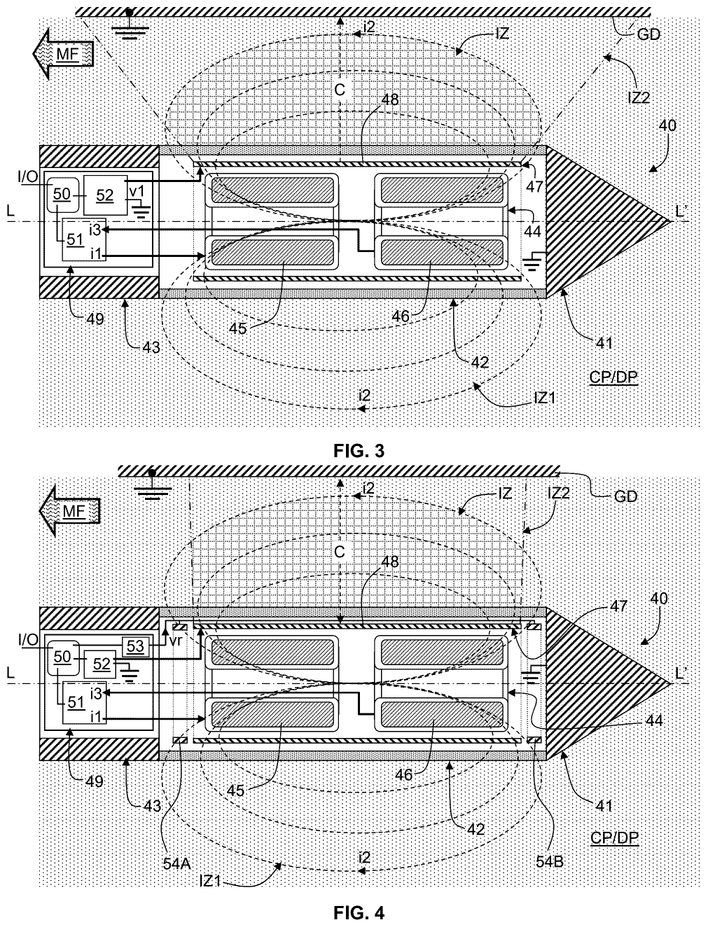

FIG. 3 is a side partial cross-sectional view illustrating a water/oil emulsion sensor 40 of the invention according to a first embodiment. FIG. 3 depicts the water/oil emulsion sensor 40 inserted into a multiphase flow mixture MF wherein the liquid dispersed phase DP (irrespective of being oil or water) is dispersed in the liquid continuous phase CP (irrespective of being water or oil).

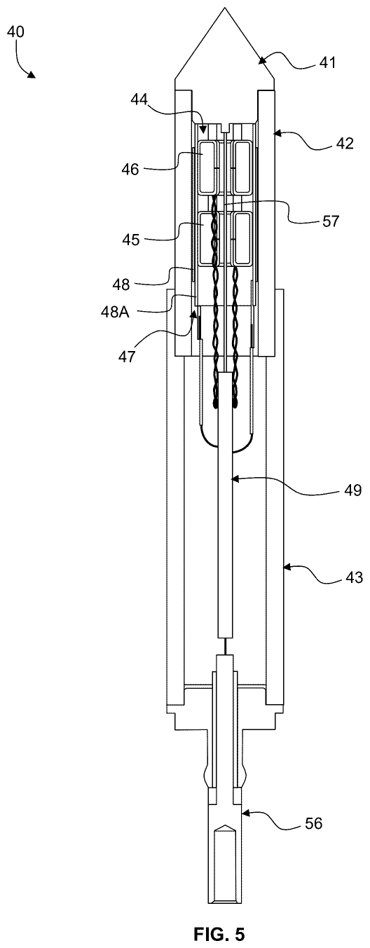

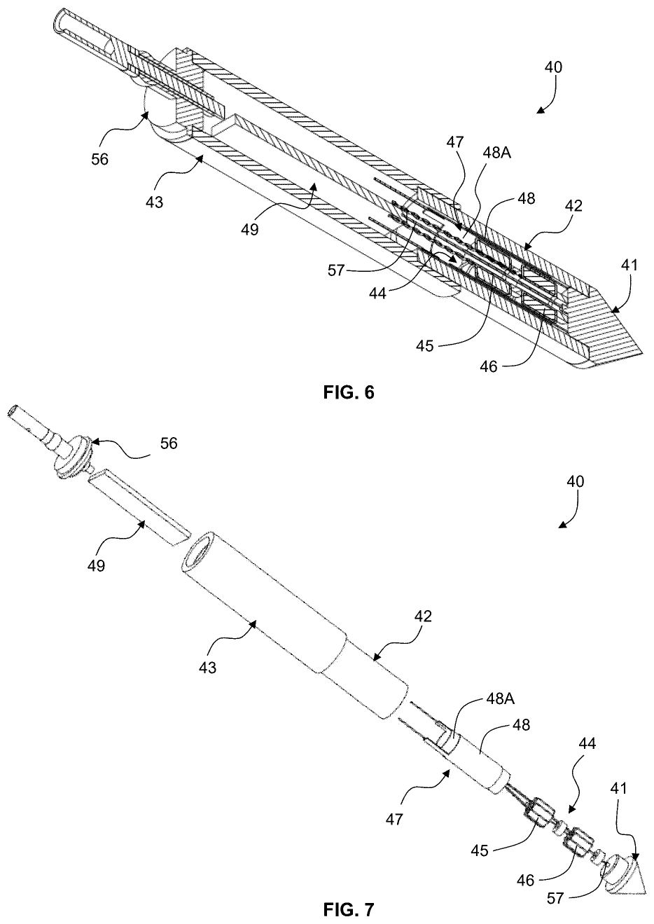

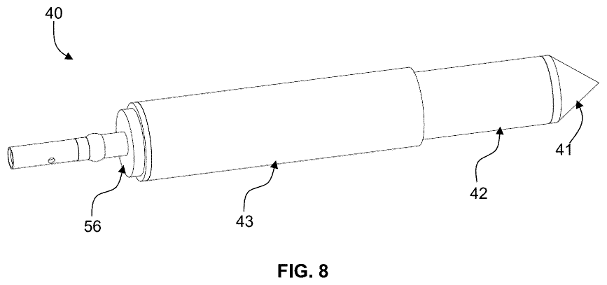

FIG. 5, FIG. 6, FIG. 7 and FIG. 8 are, respectively, a side partial cross-sectional view, a one side cross-sectional perspective view, an exploded perspective view and an assembled perspective view illustrating a practical implementation of the water/oil emulsion sensor 40. The water/oil emulsion sensor 40 comprises a conductive tip 41, a hollow isolating body 42, a hollow conductive body 43, an inductive module 44, a capacitive module 47 and an electronic unit 49. The conductive tip 41 is positioned at a front part of the sensor. The hollow isolating body 42 is positioned at a middle part of the sensor. The hollow conductive body 43 is positioned at a back part of the sensor. The front part may have a conical shape and is positioned at a distal zone of the sensor that may help avoiding, at least reducing turbulences in the flow. The middle and back parts have a substantially cylindrical shape. All these parts are coaxial and extend along a longitudinal axis and are assembled in a sealed manner.

The conductive tip 41 and the hollow conductive body 43 are made of a metallic alloy withstanding well conditions, for example nickel-chromium-based superalloy (e.g. “Inconel 718” a trademark of the company Special Metals Corporation). The hollow isolating body 42 is made of ceramic. The diameter dimensions of all these components are of the millimeter order in order to be adapted to the structure of the emulsion wherein the dispersed phase may be approximated by spheres, each sphere having a size well below 1 millimeter, typically ranging from a few micrometers to a hundred of micrometers. The length dimensions of all these components are of the centimeter order.

The inductive module 44 is housed in the hollow isolating body 42. The inductive module 44 comprises an emission coil 45 and a detection coil 46.

As an example, each coil may comprise a core made of a ferrite hollow cylinder or a toroid (square toroid or circular toroid), and a winding including multiple turns of a metallic wire (as an example from eight to twenty turns). The two ends of the wire are used to inject an excitation inductive current in the emission coil 45 and to measure an induced inductive current in the detection coil 46. The excitation inductive current may be a sinusoidal, or a triangle, or a square alternating (periodic) current. The excitation inductive current may be characterized by an excitation inductive current frequency ranging, for example, from 1 kHz to 900 kHz. The excitation inductive current may be a low power current, for example having an amplitude of the milli-Ampere order, for example from 0.1 mA to 20 mA. The characteristic amplitude, frequency and phase of the excitation inductive current may be adapted to the characteristic parameters of the inductive module 44, for example the size of the coil, the number of wire turns, the type of material used for the core.

The capacitive module 47 is housed in the hollow isolating body 42. The capacitive module 47 comprises a conductive cylindrical surface 48. The conductive cylindrical surface 48 may be supported onto a supporting tube 48A (see FIG. 5). The conductive cylindrical surface 48 is made of metal, whereas the supporting tube 48A may be made of plastic or ceramic material. The conductive cylindrical surface 48 is submitted to an excitation capacitive voltage. The impedance of the capacitive module 47 is determined to infer the capacitance between the conductive cylindrical surface 48 and another conductive element representative of a ground potential GD whose gap is filled by the fluid medium to measure. The excitation capacitive voltage may be a sinusoidal, or a triangle, or a square alternating (periodic) voltage. The excitation capacitive voltage may be characterized by an excitation capacitive voltage frequency ranging, for example, from 1 kHz to 900 kHz. The excitation capacitive voltage may have, for example, an amplitude from a few hundred millivolts to a few Volts, for example, from 100 mV to 9V. The characteristic amplitude, frequency and phase of the excitation capacitive voltage may be adapted to the characteristic parameters of the capacitive module 47, for example the size of the conductive cylindrical surface 48 in terms of length, diameter, surface, and the type of material.

The excitation inductive current and the excitation capacitive voltage are independent of each other. This means that the characteristic amplitude, frequency and phase of these current, respectively voltage are optimized for the inductive module 44 and the capacitive module 47 in a separated manner. Nevertheless, in a particular embodiment, both circuits may be substantially identical.

The conductive tip 41 is connected to the ground GD by means of a tip wire 57. The tip wire 57 travels through the hollow isolating body 42 and may be coupled to the hollow conductive body 43, for example via the electronic unit 49. The tip wire 57 closes the loop between the conductive tip 41 and the conductive body 43.

The electronic unit 49 is housed in the hollow conductive body 43. The electronic unit 49 comprises a processing module 50 (see FIG. 3 and FIG. 4), an inductive power supply and measurement module 51 connected to the inductive module 44, and a capacitive power supply and measurement module 52 connected to the capacitive module 47. The inductive power supply and measurement module 51 provides electrical power, namely excitation inductive current to the emission coil 45 and measures the induced inductive current provided by the detection coil 46. It may further include features such as voltage regulation, current limiting, and overcurrent protection. The capacitive power supply and measurement module 52 provides electrical power, namely excitation capacitive voltage to the conductive cylindrical surface 48 and measures the capacitance between the conductive cylindrical surface 48 and another conductive element representative of a ground potential GD. This other conductive element may be as described in more detail hereinafter an element of the downhole tool, of the sensor or the casing disposed at a distance close to the conductive cylindrical surface 48, ranging, for example, from a few millimeters to a few centimeters. Each of the inductive power supply and measurement module 51 and the capacitive power supply and measurement module 52 includes an analog part driving amplifier to drive the coils or the capacitance and amplify the measured signal in return. They may further include features such as voltage regulation, current limiting, and overcurrent protection. The processing module 50 processes the measured data and performs necessary calculations in order to infer the water content and the oil content of the multiphase fluid mixture (MF) flowing as a water/oil emulsion. It may include an A/D conversion microcontroller and a microprocessor for data processing and analysis. The inductive and capacitance related measurements are complex impedance measurements. The processing module 50 extracts complex impedance Z values in phase and quadrature as explained hereinafter. The electronic unit 49 may further comprise a communication interface I/O. The communication interface I/O has communication capabilities to interact with the main processing module 59 of the downhole tool 1 (visible on FIG. 1 and FIG. 2).

The water/oil emulsion sensor 40 further comprises a connector 56. The connector 56 is positioned at the back of the sensor 40 and closes the hollow conductive body 43 at the side of the sensor opposite to the conductive tip 41. An electric cable (not shown) that may include power wires and communication wires connected to the communication interface I/O is coupled to the connector 56. The electric cable couples the sensor 40 to the main processing module 59 of the downhole tool 1 (see FIG. 1 and FIG. 2).

The inductive module 44 operates as follows. The inductive power supply and measurement module 51 is arranged to inject an excitation inductive current i1 into the emission coil 45 and to measure an induced inductive current i3 in the detection coil 46. The excitation inductive current is a periodic current. When the multiphase fluid mixture MF is conductive (e.g. mainly water or water as the continuous phase), a first induced inductive current i2 is generated into a first investigation zone IZ1 surrounding the inductive module 44 outside hollow isolating body 42, and a second induced inductive current i3 proportional to the first induced inductive current i2 is measured in the detection coil 46. The second induced inductive current i3 is representative of the water content within the multiphase fluid mixture MF in the first investigation zone IZ1. When the multiphase fluid mixture MF is isolating (e.g. mainly oil or gas as the continuous phase), the induced inductive currents i2 and i3 are nil. The processing module 50 together with the inductive power supply and measurement module 51 connected to the inductive module 44 determines the complex inductor impedance Zind-j.L.ω1 (where j is the symbol indicating the imaginary part; L is the inductance of the detection coil; and ω1 is the angular frequency related to the frequency f1 of the inductive current, ω1=2πf1) by measuring the amplitude and phase (i.e. angular component) of the second induced inductive current i3. In practice, the processing module 50 together with the inductive power supply and measurement module 51 connected to the inductive module 44 measures the induced current in the secondary coil (i.e. detection coil 46), which is dependent on the volume conductivity of the fluid surrounding the sensor, which is dependent on the volume fractions of water in the emulsion.

The capacitive module 47 operates as follows. The capacitive power supply and measurement module 52 is arranged to apply an excitation capacitive voltage v1 to the conductive cylindrical surface 48 and to measure the capacitance of the capacitor composed of electrode 48 and the ground electrode GD, whose gap represents a second investigation zone IZ2 filled by the multiphase fluid mixture MF, the capacitance being representative of the oil content within the multiphase fluid mixture MF in the second investigation zone IZ2. The excitation capacitive voltage v1 may be a sinusoidal, triangular, or square voltage. More precisely, the capacitance is measured between the conductive cylindrical surface 48 and a proximate conductive element GD. When the multiphase fluid mixture MF is isolating (e.g. mainly oil or oil as the continuous phase), the dielectric permittivity of the multiphase fluid mixture MF present between the conductive cylindrical surface 48 and the proximate conductive element GD is related to the oil content of the multiphase fluid mixture MF, the quantity of water dispersed phase into the second investigation zone IZ2 will modify said permittivity, and thus the associated capacitance (i.e. the water content modifies the effective gap thickness of the capacitor and therefore the measured capacitance value will depend on the water content of the water in oil emulsion). When the multiphase fluid mixture MF is conductive (e.g. mainly water or water as the continuous phase), the capacitance is short-circuited, and no further measurement is possible with the capacitive method, it is therefore the inductive method that takes over on the other value range. The proximate conductive element GD may be a conductive element of the sensor itself, or a conductive part of the downhole tool to which the sensor is secured (e.g. a body section, an arm, etc . . . ). The processing module 50 together with the capacitive power supply and measurement module 52 connected to the capacitive module 47 determine the complex capacitor impedance Zcap=1/j.C.ω2 (where j is the symbol indicating the imaginary part; C is the capacitance medium between the conductive cylindrical surface 48 and the proximate conductive element GD; and ω2 is the angular frequency related to the frequency f2 of the capacitive voltage, ω2=2πf2) by measuring the amplitude and phase (i.e. angular component) of the capacitive voltage measured between the conductive cylindrical surface 48 and a ground GD.

The inductive module 44 is arranged in a nested and proximate manner relatively to the capacitive module 47 within the hollow isolating body 42 at the middle part of the water/oil emulsion sensor 40. The emission coil 45 and the detection coil 46 are in-line and extend according to the longitudinal axis LL′ of the sensor. The conductive cylindrical surface 48 extends according to the longitudinal axis LL′ and is coaxial to the emission coil 45 and the detection coil 46. The conductive cylindrical surface 48 is concentric and surrounds both emission and detection coils 45, 46. The diameter of the cylinder defined by the conductive cylindrical surface 48 is greater than the diameter of the cylinder defined by either the emission coil 45 or the detection coil 46. The length of the cylinder defined by the conductive cylindrical surface 48 is identical or greater than the length of the cylinder defined by both the emission and detection coils 45, 46. In other words, the cylinder defined by both the emission and detection coils 45, 46 is completely enclosed inside the cylinder defined by the conductive cylindrical surface 48. Consequently, the first and second investigation zones IZ1, IZ2 substantially overlap each other.

The inductive related measurements and the capacitance related measurements are complex impedance measurements Zind, respectively Zcap, with phase shift. The complex impedance Z depends (i.e. while not being proportional) on the nature of the multiphase fluid mixture MF and may combine a part of insulating fluid in conductive fluid (this is related to the complex inductor impedance Zind) and a part of conductive fluid in insulating fluid (this is related to the complex capacitor impedance Zcap). The processing module 50 extracts complex impedance Z values in phase (i.e. angular component) and quadrature. Therefore, the water content and the oil content of the multiphase fluid mixture MF may be estimated at a substantially identical location thus forming a more compact overlapping investigation zone IZ (i.e. the zone overlapping zones IZ1 and IZ2). Further, this combination of inductive and capacitive measurements at a substantially identical location makes it possible to continuously cover all kind of multiphase fluid mixture MF flowing as an emulsion.

Both first and second investigation zones IZ1, IZ2 may be approximated as investigation cylinders around the inductive module 44 and the capacitive module 47, each having an investigation volume in the cubic centimeter order, for example from 1 cm³ to 9 cm³, extending from two to five times the diameter of the water/oil emulsion sensor 40. The diameter of the water/oil emulsion sensor 40 may be of the millimeter order, for example a few millimeter ranging from 4 mm to 9 mm. The length of the investigation zone along the inductive module 44/the capacitive module 47 may be of the centimeter order, for example from 1 cm to 4 cm. Thus, the measurements are made in the vicinity of the water/oil emulsion sensor 40, really close to the inductive module 44/the capacitive module 47 parts, namely around the hollow isolating body 42.

In order to improve emulsion measurements accuracy, a calibration may be performed to determine the conductivity of the continuous phase (i.e. medium) because water conductivity is affected by salt content (e.g. brine is more conductive than unsalted water). This may be determined by various ways, for example sampling in the well, or in-situ measurement.

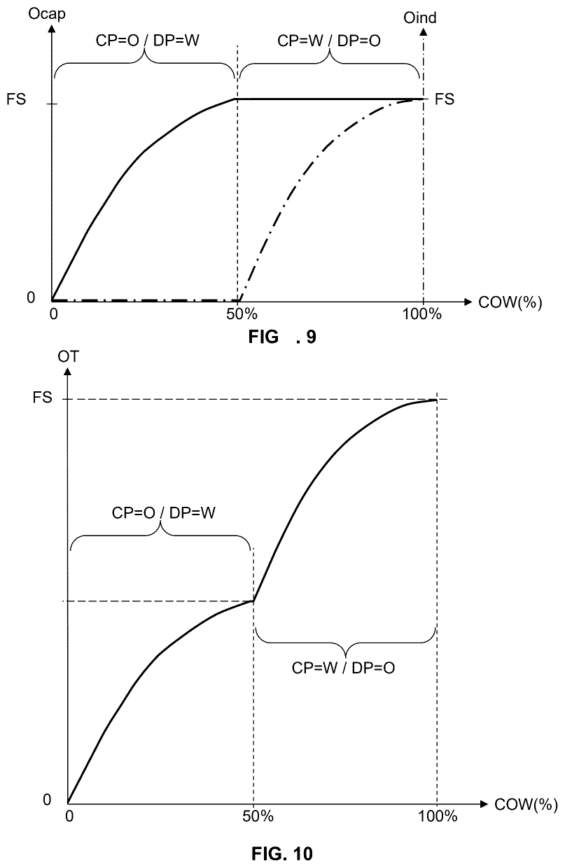

FIG. 9 and FIG. 10 are diagrams illustrating the water/oil emulsion sensor response in water/oil emulsion and how this is used to infer the water content and oil content of a multiphase fluid mixture flowing as an emulsion in a hydrocarbon well.

The diagram of FIG. 9 illustrates the capacitance output Ocap (full line) and the inductive output Oind (dash-dotted line) from zero to an arbitrary full-scale FS (ordinate) as a function of the water content in percentage COW (abscissa). From a water content COW evolving from 0% to 50% (i.e. the dispersed phase being water in the continuous phase being oil), the capacitance output Ocap (full line) increases until the capacitance output Ocap reaches a plateau at full-scale corresponding to a saturation of the capacitance measurement (i.e. 50% corresponding to a change of the continuous phase from oil to water), while the inductive output Oind (dash-dotted line) is zero (as a consequence of the medium being isolating, i.e. conductivity is zero). From a water content COW evolving from 50% to 100% (i.e. the dispersed phase being oil in the continuous phase being water), the inductive output Oind (dash-dotted line) increases until the inductive output Oind reaches a full-scale value at 100% of water, while the capacitance output Ocap (full line) stays at full scale (as a consequence of the medium being conducting, i.e. conductivity is increasing). The broken line shows when the continuous phase (medium) changes from oil (on the left of the diagram; CP=O/DP=W) to water (on the right of the diagram; CP=W/DP=O).

The diagram of FIG. 10 illustrates the response (total output OT) when the capacitance output Ocap (full line in FIG. 9) and the inductive output Oind (dash-dotted line in FIG. 9) are combined. The addition of both outputs may be performed at an analog level with an operational amplifier or digitally by the processing module 50. The result is a nonlinear response that may be approximated by a polynomial of order n. The processing module 50 of the water/oil emulsion sensor 40 determines the water content and the oil content of the multiphase fluid mixture MF flowing as an emulsion at the investigation zone IZ based on the data measured by the inductive power supply and measurement module 51 connected to the inductive module 44, and the capacitive power supply and measurement module 52 connected to the capacitive module 47. When a water/oil emulsion sensor 40 of the invention is displaced within a hydrocarbon well, the measurement log is used in the determination of water content and oil content of the water/oil emulsion. The log may be interpreted to determine sections of said well where the water/oil emulsion is more or less conductive or other sections of said well where the water/oil emulsion is more or less capacitive such that the log interpretation helps in estimating the rate of oil in the water in order to locate the oil production zones, and the rate of water in the oil in order to locate water producing zone.

FIG. 4 is a side partial cross-sectional view illustrating a water/oil emulsion sensor 40 of the invention according to a second embodiment. The second embodiment only differs from the first embodiment illustrated in FIG. 3 in that the sensor 40 further comprises an active counter electrode 54A, 54B having a first part 54A and a second part 54B on each side of the conductive cylindrical surface 48. Each of the first part 54A and second part 54B is a ring having essentially the same diameter as the conductive cylindrical surface 48. Both active counter electrode parts 54A, 54B are connected to a reverse voltage power supply 53 that supply the active counter electrode 54A, 54B with a reverse voltage vr compared to the excitation capacitive voltage v1 applied to the conductive cylindrical surface 48 by the capacitive power supply and measurement module 52. This arrangement enables focusing the field lines of the capacitive module 47 by providing field lines that are substantially straight, at least substantially more straight compared to the first embodiment. As a consequence, the overlapping investigation zone IZ according to the second embodiment is even more compact and well defined.

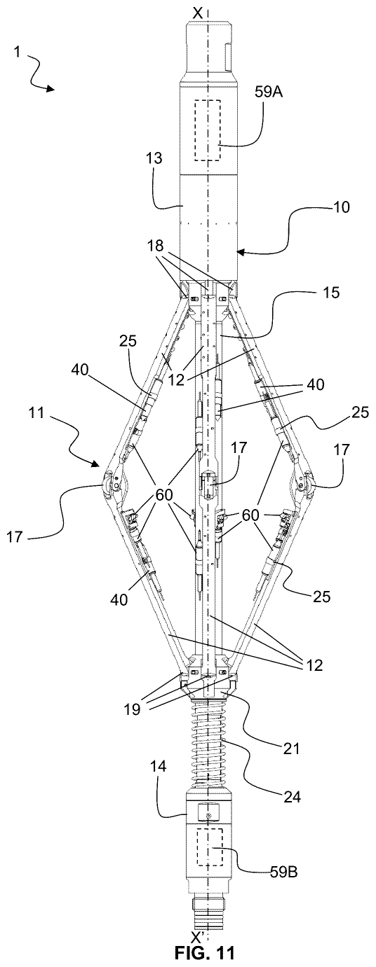

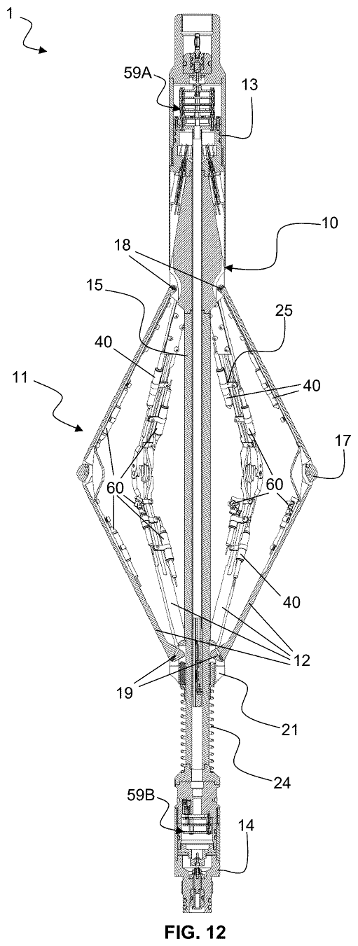

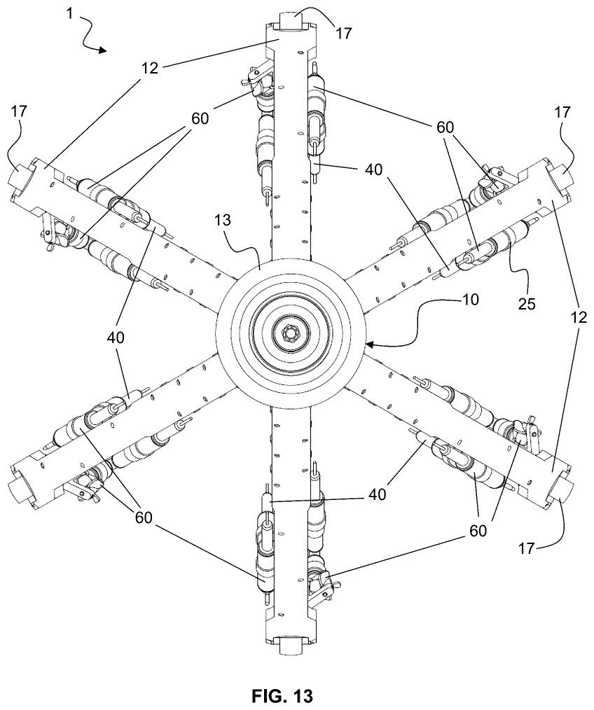

FIG. 11, FIG. 12 and FIG. 13 are, respectively, a one side perspective view, a side cross-sectional view and a front view illustrating various embodiments of a downhole tool, in particular a production logging tool 1, including an array of water/oil emulsion sensors 40 of the invention. The production logging tool 1 is intended to be deployed in any section of the hydrocarbon well, for example in the deviated well section and horizontal well section depicted in FIG. 1.

The production logging tool I has an elongated cylindrical body shape and comprises a central pressure-resistant rigid housing 10 carrying a centralizer arrangement 11. The production logging tool 1 extends longitudinally about the longitudinal axis XX′. The centralizer arrangement 11 substantially centers the production logging tool 1 with respect to the well bore axis YY′ during operations into the well bore, the longitudinal axis XX′ of the production logging tool 1 being substantially parallel, generally coaxial, coincident or mingled with the well bore axis. When the production logging tool 1 is moved along the well bore, the centralizer arrangement 11 is adapted to fit boreholes of different diameters while offering a minimal frictional resistance.

The central pressure-resistant rigid housing 10 comprises, at one end, a first housing part 13 that may include the main processing module 59 or a part 59A of the main processing module, at another end, a second housing part 14 that may include another part 59B of the main processing module and/or other electronic modules, and, centrally, a stem 15 under the form of an elongated, reduced diameter, hollow tube connecting the first and second housing parts 13, 14. The main processing module 59, 59A, 59B is used as a generic terminology herein and may provide various electronic functions, for example computations, power supply, telemetry, positioning, etc . . . . It may comprise microprocessors, power component, batteries, depth sensors and positioning sensors, etc . . . . In particular, positioning sensors may include accelerometer and gyrometer sensors which allow the measurement of tool inclination and relative bearing and, consequently, positions of downhole fluid properties analysis probes within the well section with respect to top and bottom.

The centralizer arrangement 11 comprises articulated centralizer arms 12 (six articulated centralizer arms in the present example) and associated roller hinges 17. The roller hinges 17 are positioned externally with respect to the articulated centralizer arms 12 and to the stem 15 and enter into contacting engagement with the wall 6 of the hydrocarbon well 2 (see FIG. 1 and FIG. 2). In particular, the roller hinges 17 are adapted for a smooth and low frictional drag contact with the wall 6. Each articulated centralizer arm 12 includes a first arm part and a second arm part coupled together by the associated roller hinges 17 forming an appropriate pivot connection. The first centralizer arm part and the second centralizer arm part may be identical. The centralizer arms 12 are coupled at a first side to the first housing part 13 of the housing 10 by respective pivot connection, e.g. hinges 18 and at a second side to a sliding sleeve 21 by respective pivot connection, e.g. hinges 19. The sliding sleeve 21 can slide on the stem 15. As an example, the present embodiment comprises a centralizer arrangement 11 including six centralizer arms 12. The six centralizer arms are spaced apart circumferentially about the longitudinal axis XX′ of the production logging tool 1. The six centralizer arms may be identical and equally spaced on the circumference. The centralizer arrangement 11 further comprises an axial spring element, e.g. a coil spring 24 extending around the stem 15 and being disposed in abutment between the second housing part 14 and the sliding sleeve 21.

The centralizer arrangement 11 operates as follows. The coil axial spring 24 exerts an axial force substantially along the longitudinal axis XX′ of the production logging tool 1. The axial forces act onto the sliding sleeve 21 that slides onto the stem 15. Thus, the coil spring 24 causes radial forces that act on the articulated centralizer arms 12 and associated roller hinges 17 urging them to move radially outwardly toward the wall 6 until an outmost extended position corresponding to the outermost portions of the roller hinges 17 being urged into engagement with the surface of the wall 6 (see FIG. 1). When the production logging tool 1 is run into a hydrocarbon well 4 having a diameter that changes, in particular through a restriction of smaller diameter, the wall 6 acts on the articulated centralizer arms 12 and associated roller hinges 17 that are urged to move radially inwardly towards the stem 15. This causes an inwardly oriented axial force acting onto the sliding sleeve 21 that slide onto the stem 15 in the other direction compressing the coil spring 24. In an extreme configuration, adapted to the downward movement of the tool, the articulated centralizer arms 12 and associated roller hinges 17 may be fully retracted such as being parallel to the stem 15, lying on the stem circumference surface, flush with the external surface of the first and second housing parts 13, 14. This sliding sleeve mechanism comprising the coil axial spring 24 is a passive mechanism enabling the arms 12 to be automatically radially extended such as to be deployed in the full well diameter and to engage the wall of the well. Alternatively, this passive sliding sleeve mechanism may be replaced by an active motorized mechanism, for example a motor (not shown) replacing the spring and controlling the position of the sliding sleeve. An arm opening sensor (not shown) provides a measurement of the opening of the arms 12 and enables inferring the inner diameter ID of the well section 2.

According to the present exemplary embodiment, each centralizer arm 12 may further comprise at least one, for example multiple, water/oil emulsion sensor(s) 40 secured on an internal side (the inner face facing the stem 15) or on a lateral side of the centralizer arm 12 such as to expose each water/oil emulsion sensor 40 to the multiphase fluid mixture MF flowing in the hydrocarbon well 4, and at the same time protect each sensor from a direct harmful contact with the wall 6. Each water/oil emulsion sensor 40 is secured on the corresponding centralizer arm 12 by means of appropriate probe attachments 25 at the side of centralizer arms. As an example, each probe attachment 25 may be a metal strip tightly surrounding the body of the sensor and secured to the arm by a screw and threaded hole for easy mounting and dismantling. This configuration allows reducing damage risks on the sensors during logging and/or deployment. Each water/oil emulsion sensor 40 is connected to the main processing module 59, or the main processing module parts 59A, 59B in the first housing part 13 and/or in the second housing part 14. A protective cable (not shown for sake of clarity) enclosing all the wires (power wires, communication wires, I/O wires, et . . . ) extends from the main processing module 59, 59A, 59B to the water/oil emulsion sensor 40 through pressure feedthroughs into said housing part 13, 14 and all along the centralizer arms 12. Optionally, other sensors or probes, for example downhole fluid properties analysis probe 60 of any type, namely mechanical, magnetic, optical, piezoelectric, electrical, ultrasonic, spinner or mini-spinner, etc . . . responsive to various physical entities like pressure, temperature, density, viscosity, refractive index, fluid velocity, gas bubble counts and holdups, fluorescence, spectroscopic absorption, etc . . . may be further secured to the internal side or on the lateral side of the centralizer arm 12. For example, in a particular tool configuration, the sensors or probes 60 are optical probes measuring gas holdup, mini-spinner probe measuring fluid velocity, ultrasonic doppler probe measuring fluid velocity, etc . . . The water/oil emulsion sensors 40 and the other downhole fluid properties analysis probe 60 of any type or a combination of multiple types may form an array of sensors.

Multiple water/oil emulsion sensors 40 and/or downhole fluid properties analysis probe 60 may be connected together to the main processing module 59 such as to form an array of sensors. The array of water/oil emulsion sensors and/or downhole fluid properties analysis probe 60 may be connected together such as to form a star-like arrangement of water/oil emulsion sensors. This improves the reliability because all the sensors are independently connected to the main processing module 59, one faulty sensor having no effect on another one. The array of water/oil emulsion sensors and/or downhole fluid properties analysis probe 60 may be connected together such as to form a string of sensors. This simplifies cable connection because all the sensors are connected together to the main processing module 59 via a single chain of connection.

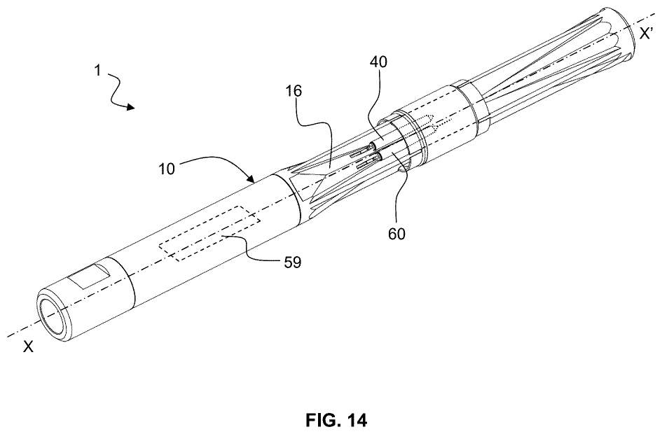

FIG. 14 is a one side perspective view illustrating another embodiment of a downhole tool, in particular a production logging tool 1, including a water/oil emulsion sensor 40 of the invention. The production logging tool 1 according to this other embodiment is intended to be deployed in a vertical section of a hydrocarbon well as depicted in FIG. 2.

The production logging tool 1 has an elongated cylindrical body shape and comprises a central pressure-resistant rigid housing 10. The production logging tool I extends longitudinally about the longitudinal axis XX′. The production logging tool 1 is coupled to surface equipment through a wireline 5 (see FIG. 2) which is operable at a surface equipment to displace the tool along the well and substantially centers the production logging tool 1 with respect to the well bore axis during operations into the well bore, the longitudinal axis XX′ of the production logging being substantially parallel, generally coincident or mingled with the well bore axis YY′ tool 1. The central pressure-resistant rigid housing 10 includes the main processing module 59 and/or other electronic modules. The central pressure-resistant rigid housing 10 comprises at least one slot 16 partially opening to the outside (i.e. towards the multiphase fluid mixture) to accommodate at least one water/oil emulsion sensor 40. Optionally, the slot 16 may also accommodate other downhole fluid properties analysis probe 60 (as described hereinbefore in relationship with the embodiments of FIG. 11, FIG. 12 and FIG. 13). Further, other similar slots 16 may be provided around the circumference of the central pressure-resistant rigid housing 10 (e.g. the depicted tool comprises four slots).

With the production logging tool of the invention, it is possible to achieve:
Accurate measurements of water and oil content of multiphase fluid mixture flowing as an emulsion whatever the configuration of the well, the well section being vertical or horizontal or having deviated well sections.
Fluid identification measurements of water/oil emulsion can be focused on a well zone with significant interest such as an oil producing zone.
Minimal perturbation of flow from tool structure, due in part to the mechanical structure of the tool.
Interchangeable sensors for maintenance issues.
Robust design allowing deployment in openhole sections.
The production logging tool structure of the invention is simple, compact achieving low cost and easy operation and maintenance.

It should be appreciated that embodiments of the production logging tool according to the present invention are not limited to the embodiment showing horizontal and vertical hydrocarbon well bores, the invention being also applicable whatever the configuration of the well bore, namely deviated or a succession of deviated and/or horizontal portions, cased or uncased. Also, the water/oil emulsion sensor of the invention is not limited to an application into a production logging tool, but can be easily adapted to various applications into analysis tools operating at downhole pressure and temperature conditions, e.g. a downhole fluid analysis tool, a wireline tool, a formation tester. Despite the fact that the illustrated production logging tool comprises only a unique measuring section, the principle of the invention would be equally applicable to a production logging tool comprises multiple measuring sections coupled together. Further, the downhole tool comprising one water/oil emulsion sensor per arm as illustrated is a non-limitative embodiment, as less of more water/oil emulsion sensors can be provided depending on the accuracy level required, the available space along the deploying arm(s), and any other consideration related to the deployment of such downhole tools in specific hydrocarbon wells. Furthermore, despite the fact that some of the drawings show sections of the hydrocarbon well full of emulsion, the invention is also applicable to perform measurement of multiphase flow in highly deviated well sections and substantially horizontal well sections (well axis YY′) wherein the multiphase fluid mixture flows as a layered flow within the well sections, for example a gas layer flowing over an emulsion of oil and water (the continuous phase being either water or oil).

1. A water/oil emulsion sensor sensitive to a water content and an oil content of a multiphase fluid mixture flowing as an emulsion in a hydrocarbon well, the water/oil emulsion sensor comprising:
a conductive tip at a front part, a hollow isolating body at a middle part and a hollow conductive body at a back part, said front part having a conical shape, said middle and back parts having a substantially cylindrical shape, said parts of said sensor being coaxial, extending along a longitudinal axis and being assembled in a sealed manner;
an inductive module housed in the hollow isolating body and comprising an emission coil and a detection coil;
a capacitive module housed in the hollow isolating body and comprising a conductive cylindrical surface; and
an electronic unit comprising a processing module coupled to an inductive power supply and measurement module connected to the inductive module, and to a capacitive power supply and measurement module connected to the capacitive module;
wherein:
the inductive power supply and measurement module is arranged to inject an excitation inductive current into the emission coil such that if the multiphase fluid mixture is conductive, a first induced inductive current is generated into a first investigation zone surrounding the inductive module and a second induced inductive current proportional to the first induced inductive current is measured in the detection coil, the second induced inductive current being representative of the water content within the multiphase fluid mixture in the first investigation zone;
the capacitive power supply and measurement module is arranged to apply an excitation capacitive voltage to the conductive cylindrical surface and to measure a capacitance of the multiphase fluid mixture in a second investigation zone surrounding the conductive cylindrical surface, the capacitance being representative of the oil content within the multiphase fluid mixture in the second investigation zone; and
the inductive module is arranged in a nested and proximate manner relatively to the capacitive module within the hollow isolating body at the middle part, the emission coil and the detection coil being in-line and extending according to the longitudinal axis, the conductive cylindrical surface being coaxial to the longitudinal axis, concentric and surrounding both emission and detection coils such that the first and second investigation zones substantially overlap each other so that the water content and oil content of the multiphase fluid mixture flowing as emulsion are determinable at a substantially identical location.

2. The water/oil emulsion sensor of claim 1, wherein the conductive tip and the hollow conductive body are made of a metallic alloy withstanding well conditions, and the hollow isolating body is made of ceramic.

3. The water/oil emulsion sensor of claim 1, wherein each of said emission coil and detection coil comprises a core made of a ferrite hollow cylinder or a toroid, and a winding including multiple turns of a metallic wire.

4. The water/oil emulsion sensor of claim 1, wherein the inductive power supply and measurement module is arranged to inject an excitation inductive current having an amplitude ranging from 0.1 mA to 20 mA and a frequency ranging from 1 kHz to 900 kHz into the emission coil.

5. The water/oil emulsion sensor of claim 1, wherein the capacitive power supply and measurement module is arranged to apply an excitation capacitive voltage having an amplitude ranging from 100 mV to 9V and a frequency ranging from 1 kHz to 900 kHz to the conductive cylindrical surface.

6. The water/oil emulsion sensor of claim 1, wherein the conductive cylindrical surface is supported onto a supporting tube, said conductive cylindrical surface being made of metal, said supporting tube being made of plastic or ceramic material.

7. The water/oil emulsion sensor of claim 1, wherein the conductive tip is connected to a ground potential by means of a tip wire, the tip wire closing a loop between the conductive tip and the hollow conductive body.

8. The water/oil emulsion sensor of claim 1, further comprising a connector positioned at the back of said sensor so as to close the hollow conductive body at a side of the sensor opposite to the conductive tip.

9. The water/oil emulsion sensor of claim 1, wherein the processing module is arranged to extract complex impedance values in angular phase and quadrature from complex inductor impedance values provided by the inductive power supply and measurement module connected to the inductive module combined with complex capacitor impedance values provided by the capacitive power supply and measurement module connected to the capacitive module and to estimate water and oil content based on an experimental model fitted by a polynomial approximation.

10. The water/oil emulsion sensor of claim 1, further comprising an active counter electrode having a first part and a second part on each side of the conductive cylindrical surface, each of said part being a ring having essentially the same diameter than the conductive cylindrical surface, both parts being connected to a reverse voltage power supply suppling the active counter electrode with a reverse voltage compared to an excitation capacitive voltage applied to the conductive cylindrical surface by the capacitive power supply and measurement module.

11. A downhole tool comprising an elongated cylindrical body of longitudinal axis, said body carrying a centralizer arrangement comprising articulated centralizer arms, said arms being operable from a retracted configuration into a radially extended configuration, wherein at least one arm carries at least one water/oil emulsion sensor sensitive to a water content and an oil content of a multiphase fluid mixture flowing as an emulsion in a hydrocarbon well, the water/oil emulsion sensor comprising:
a conductive tip at a front part, a hollow isolating body at a middle part and a hollow conductive body at a back part, said front part having a conical shape, said middle and back parts having a substantially cylindrical shape, said parts of said sensor being coaxial, extending along a longitudinal axis and being assembled in a sealed manner,
an inductive module housed in the hollow isolating body and comprising an emission coil and a detection coil,
a capacitive module housed in the hollow isolating body and comprising a conductive cylindrical surface, and
an electronic unit comprising a processing module coupled to an inductive power supply and measurement module connected to the inductive module, and to a capacitive power supply and measurement module connected to the capacitive module,
the inductive power supply and measurement module is arranged to inject an excitation inductive current into the emission coil such that if the multiphase fluid mixture is conductive, a first induced inductive current is generated into a first investigation zone surrounding the inductive module and a second induced inductive current proportional to the first induced inductive current is measured in the detection coil, the second induced inductive current being representative of the water content within the multiphase fluid mixture in the first investigation zone,
the capacitive power supply and measurement module is arranged to apply an excitation capacitive voltage to the conductive cylindrical surface and to measure a capacitance of the multiphase fluid mixture in a second investigation zone surrounding the conductive cylindrical surface, the capacitance being representative of the oil content within the multiphase fluid mixture in the second investigation zone,
the inductive module is arranged in a nested and proximate manner relatively to the capacitive module within the hollow isolating body at the middle part, the emission coil and the detection coil being in-line and extending according to the longitudinal axis, the conductive cylindrical surface being coaxial to the longitudinal axis, concentric and surrounding both emission and detection coils such that the first and second investigation zones substantially overlap each other so that water content and oil content of the multiphase fluid mixture flowing as emulsion are determinable at a substantially identical location.

12. The downhole tool of claim 11, wherein multiple water/oil emulsion sensor are connected to a main processing module such as to form an array of sensors.

13. The downhole tool of claim 11, further including other downhole fluid properties analysis probe of any type, chosen among the group comprising sensors responsive to physical parameters such as pressure, temperature, density, viscosity, refractive index, fluid velocity, gas bubble counts and holdups, fluorescence, spectroscopic absorption of the multiphase fluid mixture.

14. A downhole tool comprising an elongated cylindrical body of longitudinal axis, the body comprising at least one slot partially opening externally to accommodate at least one water/oil emulsion sensor sensitive to a water content and an oil content of a multiphase fluid mixture flowing as an emulsion in a hydrocarbon well, the water/oil emulsion sensor comprising:
a conductive tip at a front part, a hollow isolating body at a middle part and a hollow conductive body at a back part, said front part having a conical shape, said middle and back parts having a substantially cylindrical shape, said parts of said sensor being coaxial, extending along a longitudinal axis and being assembled in a sealed manner,
an inductive module housed in the hollow isolating body and comprising an emission coil and a detection coil,
a capacitive module housed in the hollow isolating body and comprising a conductive cylindrical surface, and
an electronic unit comprising a processing module coupled to an inductive power supply and measurement module connected to the inductive module, and to a capacitive power supply and measurement module connected to the capacitive module,
the inductive power supply and measurement module is arranged to inject an excitation inductive current into the emission coil such that if the multiphase fluid mixture is conductive, a first induced inductive current is generated into a first investigation zone surrounding the inductive module and a second induced inductive current proportional to the first induced inductive current is measured in the detection coil, the second induced inductive current being representative of the water content within the multiphase fluid mixture in the first investigation zone,
the capacitive power supply and measurement module is arranged to apply an excitation capacitive voltage to the conductive cylindrical surface and to measure a capacitance of the multiphase fluid mixture in a second investigation zone surrounding the conductive cylindrical surface, the capacitance being representative of the oil content within the multiphase fluid mixture in the second investigation zone,
the inductive module is arranged in a nested and proximate manner relatively to the capacitive module within the hollow isolating body at the middle part, the emission coil and the detection coil being in-line and extending according to the longitudinal axis, the conductive cylindrical surface being coaxial to the longitudinal axis, concentric and surrounding both emission and detection coils such that the first and second investigation zones substantially overlap each other so that water content and oil content of the multiphase fluid mixture flowing as emulsion are determinable at a substantially identical location.

15. The downhole tool of claim 14, wherein multiple water/oil emulsion sensor are connected to a main processing module such as to form an array of sensors.

16. The downhole tool of claim 14, further including other downhole fluid properties analysis probe of any type, chosen among the group comprising sensors responsive to physical parameters such as pressure, temperature, density, viscosity, refractive index, fluid velocity, gas bubble counts and holdups, fluorescence, spectroscopic absorption of the multiphase fluid mixture.
